(19) United States
(12) Patent Application Publication
(10) Pub. No.: US 2004/0210078 A1
(43) Pub. Date: Oct. 21, 2004

(54) Satbilisation of ethylenically-unsaturated compounds with amidoximes

(76) Inventors: Heinz Friedrich Sutoris, Worms (DE); Frank Hofer, Ludwigshafen (DE); Sylke Haremza, Neckargemund (DE); Gerhard Wagenblast, Wachenheim (DE); Volker Schliephake, Schifferstadt (DE); Ulrich Jager, Romerberg (DE); Jurgen Schroder, Ludwigshafen (DE); Harald Keller, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/486,104

(22) PCT Filed: Aug. 12, 2002
(86) PCT No.: PCT/EP02/09027

(30) Foreign Application Priority Data
Aug. 13, 2001 (DE) 10139767.4

(51) Int. Cl. C07C 255/00; C07C 69/00; C09K 3/00

(52) U.S. Cl. 558/306; 560/4; 252/1

(57) ABSTRACT

The use of amidoximes for stabilisation of ethylenically-unsaturated compounds against undesired radical polymerisation is disclosed. Combinations of amidoximes with phenol and/or phenothiazine compounds have a synergistic effect. Preferred amidoximes are nitrilotris(acetamidoxime) and nitrilotris(propionamidoxime).

The present invention relates to the use of amidoximes for inhibiting undesired free-radical polymerization of ethylenically unsaturated compounds. It further relates to a mixture comprising at least one ethylenically unsaturated compound and an effective amount of at least one amidoxime.

Chemical compounds which contain one or more ethylenically unsaturated groups have a pronounced tendency to undergo free-radical polymerization. Such compounds are used as monomers for the targeted preparation of polymers, e.g. by free-radical polymerization. At the same time, the pronounced tendency to undergo free-radical polymerization is a disadvantage in so far as undesired, spontaneous free-radical polymerization can occur both during storage and during chemical and/or physical processing, e.g. by distillation or rectification, of the ethylenically unsaturated compounds, in particular under the action of heat and/or light. Such uncontrolled free-radical polymerizations present a considerable hazard potential and frequently proceed in an explosive manner. In the distillation of mixtures comprising ethylenically unsaturated compounds, polymer formed in an uncontrolled manner can, for example, deposit on the surface of the vaporizer, where the tendency to form polymer is increased as a result of the high temperatures, and thereby cause an undesirable reduction in the heat transfer. Polymer which is formed can also block the internals in rectification columns, which causes undesirable pressure drops. Finally, the rectification process has to be interrupted to remove the polymer which has been formed.

It is therefore customary for substances which prevent or slow down spontaneous polymerization to be added to ethylenically unsaturated compounds or mixtures in which such compounds are present, both during storage and during chemical and/or physical processing. Such substances are generally referred to as inhibitors or retarders and may be summarized under the generic term “stabilizers”.

It is an object of the present invention to provide novel, effective (especially in small amounts) and/or inexpensive stabilizers which are suitable for stabilizing ethylenically unsaturated compounds against undesired free-radical polymerization.

We have found that this object is achieved by amidoximes, which are able to inhibit or retard the free-radical polymerization of ethylenically unsaturated compounds.

The invention accordingly provides a mixture comprising at least one ethylenically unsaturated compound and at least one amidoxime in an amount which is effective in inhibiting undesired polymerization.

The invention further provides a method of stabilizing materials comprising at least one ethylenically unsaturated compound against undesired free-radical polymerization by adding an effective amount of at least one amidoxime to the materials to be stabilized. The materials to be stabilized can consist exclusively of one or more ethylenically unsaturated compounds or comprise these in a concentration at which a free-radical polymerization can be spontaneously initiated.

The invention also provides for the use of amidoximes for inhibiting undesired free-radical polymerizations of ethylenically unsaturated compounds, e.g. during manufacture, storage, transport or chemical and/or physical processing.

The invention also provides a stabilizer composition which is

suitable for stabilizing ethylenically unsaturated compounds against undesired free-radical polymerization and comprises at least one amidoxime and also at least one phenol compound and/or at least one phenothiazine compound.

All statements made in respect of the mixture of the invention in the present description apply correspondingly to the method of the present invention, the stabilizer composition of the present invention and use according to the present invention, and vice versa.

For the purposes of the present invention, amidoximes are compounds which contain the tautomeric atom grouping:

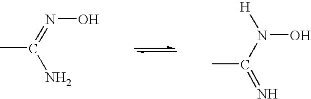

They can be prepared from nitrites by addition of hydroxylamine (cf. Houben-Weyl, Methoden der organischen Chemie, Vol. VIII, p. 692ff, and Vol. 10/4, p. 209ff). Amidoximes which can be used according to the present invention can have one or more amidoxime groups per molecule.

Suitable amidoximes have, for example, the formula:

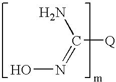

where Q is an m-valent organic radical and m is an integer from 1 to 5, preferably from 1 to 3, e.g. 2 or 3. Q is preferably an m-valent radical which is bound by a carbon and has a molecular weight of from 14 to 400.

Q is preferably hydrogen; C₁-C₂₀-alkyl, preferably C₁-C₈-alkyl; C₃-C₈-cycloalkyl, preferably C₅-C₆-cycloalkyl; C₁-C₈-alkyloxycarbonyl; mono(C₁-C₈-alkyl)aminocarbonyl or di(C₁-C₈-alkyl)aminocarbonyl; or a radical of the formula

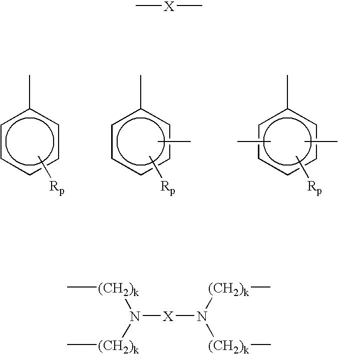

where

X is a chemical bond or C₁-C₂₀-alkylene, preferably C₂-C₈-alkylene, which may be interrupted by one, two or three groups selected from among O, S and NR′, where R′ is hydrogen, C₁-C₈-alkyl, C₃-C₈-cycloalkyl, phenyl or —(CH₂)ₖ—C(NOH)NH₂;

R is C₁-C₈-alkyl, hydroxyl, C₁-C₈-alkyloxy, amino, mono(C₁-C₈-alkyl)amino or di(C₁-C₈-alkyl)amino, fluorine or chlorine,

p is an integer from 0 to 3;

k is an integer from 1 to 3, preferably 1 or 2;

where the alkyl and alkylene groups, which may be linear or branched, may be substituted by one, two or three substituents selected from among phenyl, hydroxyl, C₁-C₈-alkoxy, phenoxy, amino, mono(C₁-C₈-alkyl)amino and di(C₁-C₈-alkyl)amino, fluorine and chlorine.

Examples of suitable amidoximes are

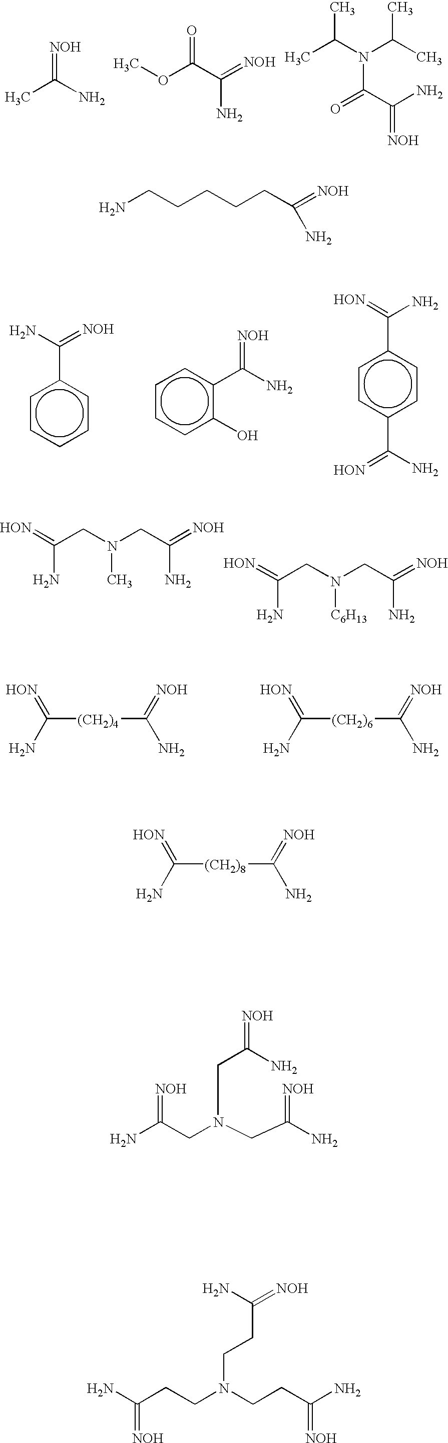

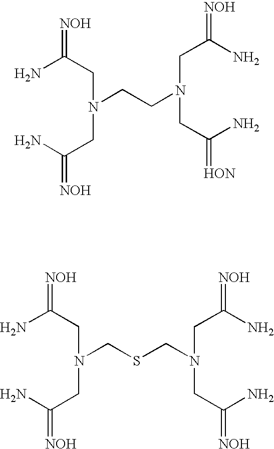

Among these, particular preference is given to nitrolotris(acetamidoxime) and nitrilotris(propionamidoxime).

The amount of amidoxime required for inhibition of undesired free-radical polymerization can easily be determined by a person skilled in the art by means of simple experiments. It depends on various factors including the reactivity of the ethylenically unsaturated compound to be stabilized, the temperature, the presence or absence of impurities which can act as free-radical initiators and the presence or absence of costabilizers. In general, from 1 to 2000 ppm by weight, frequently from 50 to 500 ppm by weight (based on the amount of ethylenically unsaturated compounds present), of amidoxime are added to the material to be stabilized.

Furthermore, it has been found that the stabilizing action of the amidoximes can be greatly increased by concomitant use of costabilizers. Particularly preferred costabilizers are phenol compounds. Possible phenol compounds are phenols having one or more hydroxyl groups and also phenols which bear further heteroatoms such as N and S on the aromatic ring. Preferred phenol compounds have one of the following structural formulae:

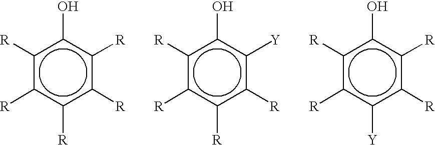

where R are each, independently of one another, hydrogen, halogen, in particular chlorine, bromine or iodine; CN, NO, NO₂, C₁-C₄-alkyl, (C₁-C₄-alkyl)carbonyl, C₂-C₄-alkenyl or C₇-C₁₀-aralkyl; and

Y is OR¹, NR²R³ or SR⁴, where R¹, R², R³ and R⁴ are each, independently of one another, hydrogen, C₁-C₈-alkyl, C₂-C₄-alkenyl, C₇-C₁₀-aralkyl, (C₁-C₄-alkyl)carbonyl or phenylcarbonyl.

Examples of suitable phenol compounds are 2-methylphenol, 4-methylphenol, 2-tert-butylphenol, 4-tert-butylphenol, 2-tert-butyl-4-methylphenol, 2,4-dimethylphenol, 2,6-dimethylphenol, 2,4-di-tert-butylphenol, 2,6-di-tert-butylphenol, 2,6-di-tert-butyl-4-methylphenol, 2-benzylphenol, 2-allylphenol, 2,6-dinitro-4-methylphenol, 2-nitro-4-methylphenol, 2,4,6-trinitrophenol, 2,4-dinitro-6-methylphenol, 2,4-dinitrophenol, 2,4-dinitro-6-sec-butylphenol, 4-cyano-2-nitrophenol, 3-iodo-4-cyano-5-nitrophenol, hydroquinone monomethyl ether, 4-tert-butylcatechol, hydroquinone, 4-methoxyphenol, 4-ethoxyphenol, 4-propoxyphenol, 4-butoxyphenol, 4-heptoxyphenol, hydroquinone monobenzyl ether, catechol, 2-methoxyphenol, 2,4-dichlorohydroquinone, 2,5-di-tert-butylhydroquinone, 2-acetylhydroquinone, hydroquinone monobenzoate, 2,3,5-trimethylhydroquinone, 4-aminophenol, 2-aminophenol, 2-N,N-dimethylaminophenol, 2-mercaptophenol, 4-mercaptophenol, catechol monobutyl ether, 4-ethylaminophenol, 2,3-dihydroxyacetophenone and 2-methylthiophenol.

Further suitable phenol compounds are n-octadecyl β-(3,5-di-tert-butyl-4-hydroxyphenyl)propionate, 1,1,3-tris(2-methyl-4-hydroxy-5-tert-butylphenyl)butane, 1,3,5-trimethyl-2,4,6-tris(3,5-di-tert-butyl-4-hydroxybenzyl) benzene, 1,3,5-tris(3,5-di-tert-butyl-4-hydroxybenzyl) isocyanurate, 1,3,5-tris[β-(3,5-di-tert-butyl-4-hydroxyphenyl)propionyloxyethyl]isocyanurate, 1,3,5-tris(2,6-dimethyl-3-hydroxy-4-tert-butylbenzyl) isocyanurate, pentaerythrityl tetrakis[β-(2,5-di-tert-butyl-4-hydroxyphenyl)propionate].

Preferred phenol compounds are 4-methoxyphenol, 2-methoxyphenol 2,4-di-tert-butylphenol, 2,6-di-tert-butyl-4-methylphenol and 2-tert-butylphenol.

The phenol compound is generally used in an amount of from 5 to 200% by weight, preferably from 25 to 100% by weight, based on the amidoxime.

Further preferred costabilizers are phenothiazine compounds. Suitable phenothiazine compounds are phenothiazine, bis(α-methylbenzyl)phenothiazine, 3,7-dioctylphenothiazine, bis(α-dimethylbenzyl)phenothiazine, N-hydroxyphenothiazine, 2-methoxyphenothiazine, phenothiazine 5-oxide, 2-(trifluoromethyl)phenothiazine, 2-acetylphenothiazine, 2-ethylthiophenothiazine. Phenothiazine is preferred.

The phenothiazine compound is generally used in an amount of from 5 to 50% by weight, preferably from 10 to 30% by weight, based on the amidoxime.

The combination of at least one amidoxime and at least one phenol compound, the combination of at least one amidoxime and at least one phenothiazine compound and the combination of at least one amidoxime, at least one phenol compound and at least one phenothiazine compound display a synergistic action in the stabilization of ethylenically unsaturated compounds against undesired free-radical polymerization. Particularly preferred embodiments of the invention are mixtures comprising

at least one ethylenically unsaturated compound, at least one amidoxime and at least one phenol compound; or

at least one ethylenically unsaturated compound, at least one amidoxime and at least one phenothiazine compound; or

at least one ethylenically unsaturated compound, at least one amidoxime, at least one phenol compound and at least one phenothiazine compound.

The mixture of the present invention may further comprise additional constituents which inhibit free-radical polymerization. Examples of such other polymerization inhibitors are organic nitro or nitroso compounds such as 1,3-dinitrobenzene, 1,4-dinitrobenzene, 2,4-dinitrochlorobenzene, N-nitrosoarylamines, or nitrosobenzene. A further polymerization inhibitor is p-phenylenediamine.

Ethylenically unsaturated compounds which can be stabilized according to the present invention include, inter alia, compounds such as olefins, e.g. isobutene, ethylene, propylene, vinylaromatic monomers such as styrene, α-methylstyrene, o-chlorostyrene or vinyltoluenes, conjugated C₄-C₈-dienes such as butadiene or isoprene, esters of vinyl alcohol and monocarboxylic acids having from 1 to 18 carbon atoms, e.g. vinyl acetate, vinyl propionate, vinyl n-butyrate, vinyl laurate and vinyl stearate.

The method of stabilization of the present invention is particularly useful for α,β-monoethylenically unsaturated monocarboxylic and dicarboxylic acids having from 3 to 6 carbon atoms, especially acrylic acid, methacrylic acid, maleic acid, fumaric acid and itaconic acid, esters of the abovementioned carboxylic acids and alkanols having from 1 to 12, frequently from 1 to 8 and often from 1 to 4, carbon atoms, in particular methyl, ethyl, n-butyl, isobutyl, tert-butyl and 2-ethylhexyl acrylate and methacrylates, dimethyl maleate and di-n-butyl maleate. The precursor aldehydes, nitriles and amides of the abovementioned α,β-monoethylenically unsaturated monocarboxylic and dicarboxylic acids having from 3 to 6 carbon atoms, e.g. acrolein, methacrolein, acrylonitrile, methacrylonitrile, acrylamide and methacrylamide, can also be stabilized by the method of the present invention. The stabilization method of the present invention is also applicable to monomers such as vinylsulfonic acid and N-vinylpyrrolidone.

Amidoximes are, either alone or in combination with costabilizers, suitable both for storage stabilization and for process stabilization in the preparation, purification and chemical reaction of ethylenically unsaturated compounds. Purification can be carried out by, for example, distillation at from 50 to 300° C., frequently from 50 to 200° C. or else from 50 to 150° C. Ethylenically unsaturated compounds are typically stored at low to slightly elevated temperatures, in general at ambient temperature, e.g. from 0 to 40° C.

According to the present invention, amidoximes, either alone or in combination with costabilizers, are particularly useful for effecting stabilization in the treatment of (meth)acrylic esters by distillation (rectification) when they are being separated by distillation or rectification from product mixtures obtained in the acid-catalyzed esterification of (meth)acrylic acid with alcohols, in particular C₁-C₁₂- or C₁-C₈-alkanols, before and/or after removal of the acid catalyst.

The stabilization of a mixture comprising (meth)acrylic esters and being subjected to a distillation (rectification) can be effected in a simple fashion by adding an amidoxime, either alone or in combination with costabilizers, prior to the distillation (rectification). The addition can also be made to the feed to the distillation (rectification) column. Inhibitor can also, either additionally or as an alternative, be added at the top of the column to effect stabilization.

The amidoxime and, if used, the costabilizers can be added in succession, simultaneously or in premixed form.

When the stabilization method of the present invention is employed, molecular oxygen or a mixture thereof with an inert gas, e.g. air, can be passed through the distillation (rectification) columns.

The amidoxime and the costabilizers can be added at various addition points. Thus, for example, components can be introduced at the top of the rectification column and other components can be introduced into the bottom of and/or the feed to the rectification column. This applies, for example, to rectifications in which the (meth)acrylic ester is separated off at the top, at the bottom and/or via a side offtake. It may also be advantageous in the case of a continuous distillation (rectification) of (meth)acrylic esters for at least one component to be added only from time to time, i.e. periodically (e.g. at the top of the column, at the bottom and/or to the feed).

The above statements regarding stabilization according to the present invention in the separation of (meth)acrylic esters from acid-catalyzed esterification mixtures by distillation (rectification) apply analogously to separation of (meth)acrylic acid or (meth)acrolein from mixtures in which they are present by distillation (rectification).

(Meth)acrylic acid is, for example, obtainable by catalytic gas-phase oxidation of alkanes, alkanols, alkenes or alkenals containing 3 or 4 carbon atoms. (Meth)acrylic acid can be obtained particularly advantageously by, for example, catalytic gas-phase oxidation of propane, propene, tert-butanol, isobutene, isobutane, isobutyraldehyde or methacrolein. However, compounds from which the actual C₃-/C₄ starting compound is formed only as an intermediate during the gas-phase oxidation are also possible as starting materials. An example which may be mentioned is the methyl ether of tert-butanol.

These starting gases, generally diluted with inert gases such as nitrogen, CO, CO₂, saturated hydrocarbons and/or steam, are passed in admixture with oxygen at elevated temperatures (usually from 200 to 400° C.) and atmospheric or superatmospheric pressure over mixed oxide catalysts comprising transition metals (e.g. Mo, V, W and/or Fe) and converted by oxidation into (meth)acrylic acid.

The catalytic gas-phase oxidation generally gives a reaction gas mixture which consists essentially of (meth)acrylic acid, the inert diluent gases and by-products and from which the (meth)acrylic acid has to be separated. Apart from by-products which are comparatively simple to remove, e.g. acetic acid, the reaction gas mixture frequently also contains lower aldehydes which are closely related to (meth)acrylic acid and are therefore difficult to separate off, e.g. formaldehyde, acetaldehyde, acrolein, methacrolein, propionaldehyde, n-butyraldehyde, benzaldehyde, furfural and crotonaldehyde, and also possibly maleic anhydride. Based on the amount of (meth)acrylic acid present in the reaction gas mixture, the total amount of by-products is generally ≦2% by weight, usually ≧0.05% by weight.

The (meth)acrylic acid can advantageously be separated from the reaction gas mixture from the catalytic gas-phase oxidation by countercurrent absorption using a high-boiling inert liquid and subsequent work-up by distillation (cf. DE-A 21 36 396 and DE-A 43 08 087). The amidoxime and any costabilizers used can be injected directly into the gas stream or can be added beforehand to the high-boiling liquid. As an alternative, absorption can be carried out using water/aqueous acrylic acid in countercurrent, followed by extractive or azeotropic distillation (cf. EP-B 0 009 545, U.S. Pat. No. 5,154,800, DE-A 34 29 391 and DE-A 21 21 123).

Possible high-boiling inert liquids include, inter alia, biphenyl, diphenyl ether and dimethyl phthalate and also mixtures thereof.

The process can be carried out essentially as follows: the reaction gas mixture is passed through an absorption column in countercurrent to the descending absorption liquid, the volatile secondary components are then substantially removed from the liquid output from the absorption column, which is composed essentially of (meth)acrylic acid, the absorption medium and secondary components, by stripping with inert gas in a desorption column, and the liquid output from the desorption column, which comprises (meth)acrylic acid and the absorption medium as main constituents, is subsequently treated by rectification to separate off crude (meth)acrylic acid.

As an alternative, the (meth)acrylic acid can firstly be absorbed in water from the reaction gases from the catalytic gas-phase oxidation and the water can subsequently be removed from the aqueous mixtures comprising (meth)acrylic acid by rectification after addition of an organic azeotropic entrainer.

There is also a need for effective stabilization against premature polymerization in the preparation of pure acrylic acid (purity: >99.7% by weight) from crude acrylic acid (purity: >99% by weight) by rectification.

According to the present invention, amidoximes can be employed for stabilization in all the abovementioned rectification tasks. The method of stabilization according to the present invention is also advantageous in the separation of mixtures comprising (meth)acrylic acid or esters thereof by crystallization.

(Meth)acrolein can be obtained in a corresponding manner by catalytic gas-phase oxidation in which the oxidation is carried out only to the first oxidation stage. The (meth)acrolein present in the reaction gas mixture is generally firstly separated off from the reaction gas mixture by extraction with water and is subsequently isolated from the aqueous solution by distillation (rectification). According to the present invention, amidoximes are suitable for all the process steps mentioned.

In general, the amount of amidoximes and any costabilizers used are chosen so that they are completely soluble in the substance to be stabilized. They are frequently added not as pure substances but as a suspension, emulsion or solution. Suitable solvents and/or dispersion media are, in particular, those substances which are constituents of the system to be stabilized, e.g. in the case of chemical reactions such as esterifications, starting materials or products of the reaction, and in the case of extractions, especially the extraction medium or a component thereof.

The invention is illustrated by the following example.

### EXAMPLE

2 ml of acrylic acid are admixed with various polymerization inhibitors and in each case sealed in an air-saturated state into a glass ampoule (20 ml capacity). The gastight glass ampoule was then dipped into an oil bath at 125° C. to such a depth that the surface of the liquid acrylic acid and the surface of the oil bath were at the same height. The time until the acrylic acid had fully polymerized was then determined (by visually observing the time at which the acrylic acid had solidified).

The following table shows the inhibitors used and the results obtained (amounts in ppm by weight, based on the amount of acrylic acid).

The following amidoxime 1 was tested:

TABLE 1

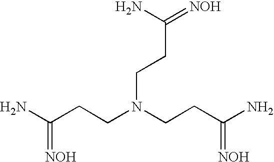

| | | | Hydroqui- | |
|---|---|---|---|---|
| | | | none mono- | Time to |
| Experi- | Phenothia- | | methyl | complete |
| ment | zine | Amidoxime 1 | ether | polymerization |
| No. | (ppm) | (ppm) | (ppm) | (min) |
| 1 unstab. | — | — | — | 8 |
| 2 | — | 100 | — | 22 |
| 3 | 20 | — | — | 90 |
| 4 | 20 | 50 | | 150 |
| 5 | 20 | 100 | | 175 |
| 6 | 20 | 200 | | 221 |
| 7 | | | 50 | 49 |
| 8 | | 100 | 50 | 80 |
| 9 | 20 | | 50 | 170 |
| 10 | 20 | 50 | 50 | 184 |
| 11 | 20 | 100 | 50 | 202 |
| 12 | 20 | 200 | 50 | 275 |

1. A mixture comprising at least one ethylenically unsaturated compound and at least one amidoxime in an amount which is effective in inhibiting undesired free-radical polymerization.

2. A mixture as claimed in claim 1, wherein the amidoxime is selected from among nitrilotris(acetamidoxime) and nitrilotris(propionamidoxime).

3. A mixture as claimed in claim 1 which further comprises a phenol compound.

4. A mixture as claimed in claim 3, wherein the phenol compound is selected from among 4-methoxyphenol, 2,4-di-tert-butylphenol, 2,6-di-tert-butyl-4-methylphenol and 2-tert-butylphenol.

5. A mixture as claimed in claim 1 which further comprises a phenothiazine compound.

6. A mixture as claimed in claim 1, wherein the ethylenically unsaturated compound is selected from among acrylic acid, methacrylic acid, acrolein, methacrolein, vinylaromatic compounds, acrylic esters, methacrylic esters, acrylonitrile, methacrylonitrile, acrylamide, methacrylamide, olefins, conjugated C₄-C₈-dienes and mixtures thereof.

7. A method of stabilizing materials comprising at least one ethylenically unsaturated compound against undesired free-radical polymerization by adding at least one amidoxime to the materials to be stabilized.

8. The use of amidoximes for inhibiting undesired free-radical polymerization of ethylenically unsaturated compounds.

9. A stabilizer composition comprising at least one amidoxime and also at least one phenol compound and/or at least one phenothiazine compound.
